(19) United States
(12) Patent Application Publication
(10) Pub. No.: US 2010/0240754 A1
(43) Pub. Date: Sep. 23, 2010

(54) UNSATURATED FATTY AMINO ACID DERIVATIVES AND USE THEREOF IN DERMAL COSMETOLOGY

(75) Inventors: Roger Tarroux, Toulouse (FR); Marie Charveron, Toulouse (FR); Sylvie Daunes-Marion, Toulouse (FR); Natacha Frison, Creil (FR); Benôit Folleas, Senlis (FR); Jean-Louis Brayer, Nanteuil Le Haudoin (FR)

Correspondence Address: BIRCH STEWART KOLASCH & BIRCH, PO BOX 747, FALLS CHURCH, VA, 22040-0747, US

(73) Assignee: Pierre Fabre Demo-Cosmetique, Boulogne-billancourt (FR)

(21) Appl. No.: 12/523,231

(22) PCT Filed: Jan. 16, 2008
(86) PCT No.: PCT/EP08/50471

§ 371 Date: Nov. 24, 2009

(30) Foreign Application Priority Data
Jan. 16, 2007 (FR) 0700291
Mar. 15, 2007 (FR) 0753864

(51) Int. Cl. C07C 229/30; C07C 227/00; A61K 31/197; A61K 8/44; A61Q 19/08; A61P 17/00

(52) U.S. Cl. 514/561; 554/103; 554/114; 560/172; 562/574

(57) ABSTRACT

The present invention relates to drugs consisting of unsaturated fatty amino-acid derivatives of the general formula (I), and to their pharmaceutically acceptable acid addition salts, in which: X is oxygen or NH, Rn are independently hydrogen or a (C₁-C₆)alkyl optionally substituted by halogen; R₁ is hydrogen, fluorine, chlorine or bromine, or a CF₃ or CHF₂ or a (C₁-C₆)alkyl, (C₁-C₆)alkenyl or (C₁-C₆)alkynyl, optionally substituted by one or more halogen atoms; R is hydrogen or a (C₁-C₆)alkyl or (C₃-C₆)cycloalkyl optionally substituted by one or more halogen atoms; Ra and Rb are independently hydrogen (C₁-C₆)alkyl or (C₁-C₆)acyl, and Ra and Rb a hydrocarbonated cycle containing 4 to 6 carbon atoms; and n is an integer between 2 and 14.

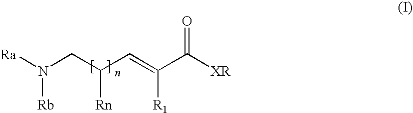

The object of the present invention is new unsaturated fatty amino acids as well as their use in dermal cosmetology.

More particularly, the present invention relates to unsaturated fatty amino acid derivatives, as drugs, fitting the general formula (A):

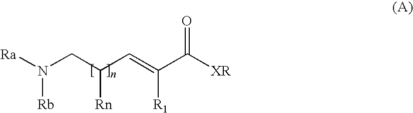

as well as their pharmaceutically acceptable acid addition salts, wherein:
X represents an oxygen or an NH group;
Rn represent independently of each other a hydrogen atom or a linear or branched alkyl group comprising 1-6 carbon atoms, and being optionally substituted with one or more halogen atoms, in particular fluorine;
R₁ represents a hydrogen, fluorine, chlorine or bromine atom, or else a —CF₃ or —CHF₂ group or else a linear or branched alkynyl, alkenyl or alkyl group comprising 1-6 carbon atoms, and being optionally substituted with one or more halogen atoms, in particular fluorine, and/or with one or more oxygen, nitrogen and/or sulfur atoms;
R represents a hydrogen atom or a linear or branched alkyl group comprising 1-6 carbon atoms, or a cycloalkyl group comprising 3-6 carbon atoms, and being optionally substituted with one or more halogen atoms, in particular fluorine and/or with one or more oxygen and/or nitrogen atoms;
Ra and Rb represent independently of each other a hydrogen atom or a linear or branched alkyl or acyl group comprising 1-6 carbon atoms, optionally substituted with one or more oxygen and/or nitrogen atoms, Ra and Rb may form together a hydrocarbon cycle including 4-6 carbon atoms, optionally substituted with one or more oxygen and/or nitrogen atoms; and
n is an integer comprised between 2 and 14.

By “alkyl” group, is meant a linear or branched saturated hydrocarbon chain, preferably including 1-6 carbon atoms, such as for example a methyl, ethyl, isopropyl, tertio-butyl, pentyl group, etc.

By “alkenyl” group, is meant a linear or branched hydrocarbon chain including at least one double bond and preferably including 2-6 carbon atoms, such as for example an ethenyl, propenyl, 2,4-hexadienyl group, etc.

By “alkynyl” group, is meant a linear or branched hydrocarbon chain including at least one triple bond and preferably including 2-6 carbon atoms, such as for example an ethynyl, propynyl, 2,4-hexadiynyl group, etc.

By “cycloalkyl group” group, is meant a saturated cyclic hydrocarbon group, preferably including 3-6 carbon atoms, such as for example a cyclopropyl, cyclohexyl, cyclopentyl group, etc.

By “acyl” group, is meant an alkyl-carbonyl group, i.e. an alkyl group as defined above bound via a carbonyl group, such as for example an acetyl.

By “halogen”, is meant fluorine, bromine, iodine or chlorine.

The present invention in particular relates to unsaturated fatty amino acid derivatives as drugs, fitting the general formula (I):

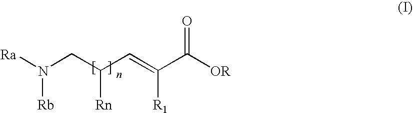

as well as to their pharmaceutically acceptable addition acid salts, wherein:
Rn represent independently of each other a hydrogen atom or a linear or branched alkyl group comprising 1-6 carbon atoms, and being optionally substituted with one or more halogen atoms, in particular fluorine;
R₁ represents a hydrogen, fluorine, chlorine or bromine atom, or else a —CF₃ or —CHF₂ group or else a linear or branched alkyl, alkenyl or alkynyl group, comprising 1-6 carbon atoms, and being optionally substituted with a halogen atom, in particular fluorine;
R represents a hydrogen atom or a group R′ representing a linear or branched alkyl group comprising 1-6 carbon atoms, or a cycloalkyl group comprising 3-6 carbon atoms, and being optionally substituted with a halogen atom, in particular fluorine;
Ra and Rb represent independently of each other a hydrogen atom or a linear or branched alkyl or acyl group comprising 1-6 carbon atoms, Ra and Rb may form together a hydrocarbon cycle including 4-6 carbon atoms; and
n is an integer comprised between 2 and 14.

According to a particular feature of the invention, the unsaturated fatty amino acid derivatives of general formula (A) or (I) meet the following criteria:
Rn represent a hydrogen atom on all the carbon atoms of the carbon chain except one where Rn represents a linear or branched alkyl group comprising 1-6 carbon atoms, and being optionally substituted with one or more halogen atoms, in particular fluorine;
R represents a hydrogen atom or a linear or branched alkyl group comprising 1-6 carbon atoms, or a cycloalkyl group comprising 3-6 carbon atoms, and being optionally substituted with one or more halogen atoms, in particular fluorine; R preferably represents a hydrogen atom;
n, Ra, Rb and R₁ are as defined earlier.

According to a particular feature of the invention, the unsaturated fatty amino acid derivatives of general formula (A) or (I) meet the following criteria:
Ra and Rb represent a hydrogen atom.

According to a particular feature of the invention, the unsaturated fatty amino acid derivatives of general formula (A) or (I) meet the following criteria:
R represent a hydrogen atom.

According to a particular feature of the invention, the unsaturated fatty amino acid derivatives of general formula (A) or (I) meet the following criteria:
Rn represent a hydrogen atom.

According to a particular feature of the invention, the unsaturated fatty amino acid derivatives of general formula (A) or (I) meet the following criteria:
R₁ represents a hydrogen or fluorine atom.

According to a particular feature of the invention, the unsaturated fatty amino acid derivatives of general formula (A) or (I) meet the following criteria:
n is comprised between 3 and 5.

According to a particular feature of the invention, the unsaturated fatty amino acid derivatives of general formula (A) or (I) meet the following criteria:
n is comprised between 9 and 14.

According to a particular feature of the invention, the unsaturated fatty amino acid derivatives of general formula (A) or (I) meet the following criteria:
n is equal to 3.

The unsaturated fatty amino acid derivatives of general formula (A) or (I), as defined earlier, may exist as Z or E isomers or as a mixture of Z isomers and E isomers, in any amounts.

In the sense of the present invention, the term “pharmaceutically acceptable addition acid salts” means salts formed by addition of an acid on the compound, which are non-toxic and which have the pharmacological activity of the parent compound.

The acid addition salts may be formed from inorganic acids such as hydrochloric acid, hydrobromic acid, sulfuric acid, nitric acid or phosphoric acid, or else from organic acids, such as acetic acid, benzene-sulfonic acid, benzoic acid, camphor-sulfonic acid, citric acid, ethane-sulfonic acid, fumaric acid, glucoheptonic acid, gluconic acid, glutamic acid, glycolic acid, hydroxynaphthoic acid, 2-hydroxyethane-sulfonic acid, lactic acid, maleic acid, malic acid, mandelic acid, methane-sulfonic acid, muconic acid, 2-naphthalene-sulfonic acid, propionic acid, salicylic acid, succinic acid, dibenzoyl-L-tartaric acid, tartaric acid, p-toluene-sulfonic acid, trimethylacetic acid or trifluoroacetic acid.

The preferred pharmaceutically acceptable salts are salts formed from hydrochloric acid.

The compounds, object of the present invention, preferably exist as hydrochloride salts.

Advantageously, the unsaturated fatty amino acid derivatives of the invention are selected from:
(E)-6-amino-hex-2-enoic acid hydrochloride,
(E)-7-amino-hept-2-enoic acid hydrochloride,
(E)-8-amino-oct-2-enoic acid hydrochloride,
(E)-9-amino-non-2-enoic acid hydrochloride,
(E)-10-amino-dec-2-enoic acid hydrochloride,
(E)-14-amino-tetradec-2-enoic acid hydrochloride,
6-amino-2-fluoro-hex-2-enoic acid hydrochloride as a mixture of Z and E isomers,
(Z)-6-amino-2-fluoro-hex-2-enoic acid hydrochloride,
(E)-6-amino-2-fluoro-hex-2-enoic acid hydrochloride,
7-amino-2-fluoro-hept-2-enoic acid hydrochloride as a mixture of Z and E isomers,
(Z)-7-amino-2-fluoro-hept-2-enoic acid hydrochloride,
9-amino-2-fluoro-non-2-enoic acid hydrochloride,
10-amino-2-fluoro-dec-2-enoic acid hydrochloride and
14-amino-2-fluoro-tetradec-2-enoic acid hydrochloride.

The present invention also relates to unsaturated fatty amino acid derivatives, as novel chemical compounds, of general formula (A):

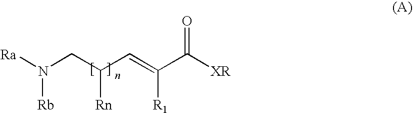

as well as their pharmaceutically acceptable acid addition salts, wherein:
X represents an oxygen or an NH group;
Rn represent independently of each other a hydrogen atom or a linear or branched alkyl group comprising 1-6 carbon atoms, and being optionally substituted with one or more halogen atoms in particular fluorine;
R₁ represents a hydrogen, fluorine, chlorine or bromine atom, or else a —CF₃ or —CHF₂ group or else a linear or branched alkynyl, alkenyl or alkyl group, comprising 1-6 carbon atoms, and being optionally substituted with one or more halogen atoms, in particular fluorine, and/or with one or more oxygen, nitrogen and/or sulfur atoms;
R represents a hydrogen atom or a linear or branched alkyl group comprising 1-6 carbon atoms, or a cycloalkyl group comprising 3-6 carbon atoms, and being optionally substituted with one or more halogen atoms, in particular fluorine, and/or with one or more oxygen and/or nitrogen atoms;
Ra and Rb represent independently of each other a hydrogen atom or a linear or branched alkyl or acyl group comprising 1-6 carbon atoms, optionally substituted with one or more oxygen and/or nitrogen atoms, Ra and Rb may form together a hydrocarbon cycle including 4-6 carbon atoms; optionally substituted with one or more oxygen and/or nitrogen atoms; and
n is an integer comprised between 2 and 14.
   the following compounds being excluded: 6-amino-hex-2-enoic acid, its hydrochloride and its trifluoroacetate, 8-amino-oct-2-enoic acid trifluoroacetate, 8-(dimethylamino)-oct-2-enoic acid, 12-(dimethylamino)-dodec-2-enoic acid, ethyl 6-(isopropylamino)-2-methyl-hex-2-enoate, ethyl 6-(tert-butylamino)-2-methyl-hex-2-enoate, methyl 6-amino-2-methyl-hex-2-enoate, methyl 7-amino-kept-2-enoate, methyl 7-amino-2-methyl-hept-2-enoate and methyl 7-amino-4-methyl-hept-2-enoate, as well as, when Ra and Rb are substituted with one or more oxygen atoms, methyl 6-(2-ethoxy-5-oxo-pyrrolidin-1-yl)-hex-2-enoate and methyl 7-(2-ethoxy-5-oxo-pyrrolidin-1-yl)-hept-2-enoate.

According to a particular characteristic of the invention, the fatty unsaturated amino acids of the invention, as new chemical compounds, fitting the general formula (I):

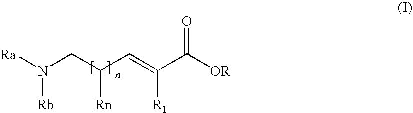

as well as their pharmaceutically acceptable acid addition salts, wherein:
Rn represent independently of each other a hydrogen atom or a linear or branched alkyl group comprising 1-6 carbon atoms, and being optionally substituted with one or more halogen atoms, in particular fluorine;
R₁ represents a hydrogen, fluorine, chlorine or bromine atom, or else a —CF₃ or —CHF₂ group or else a linear or branched alkynyl, alkenyl or alkyl group, comprising 1-6 carbon atoms, and being optionally substituted with a halogen atom, in particular fluorine;
R represents an hydrogen atom or an R′ group representing a branched or linear alkyl group, comprising 1-6 carbon atoms, or a cycloalkyl group comprising 3-6 carbon atoms, and being optionally substituted with an halogen atom, in particular fluorine;
Ra and Rb independently of each other represent an hydrogen atom or a linear or branched alkyl or acyl group, comprising 1-6 carbon atoms, Ra and Rb together may form an hydrocarbon group including 4-6 carbon atoms; and
n is en integer comprised between 2 and 14;
   the following compounds being excluded: 6-amino-hex-enoic acid, its hydrochloride and its trifluoroacetate, 8-amino-oct-2-enoic acid trifluoroacetate, 8-(dimethylamino)-oct-2-enoic acid, 12-(dimethylamino)-dodec-2-enoic acid, ethyl 6-(isopropylamino)-2-methyl-hex-2-enoate, ethyl 6-(tert-butylamino)-2-methyl-hex-2-enoate, methyl 6-amino-2-methyl-hex-2-enoate, methyl 7-amino-hept-2-enoate, methyl 7-amino-2-methyl-hept-2-enoate and methyl 7-amino-4-methyl-hept-2-enoate.

According to a particular feature of the invention, the unsaturated fatty amino acid derivatives of general formula (A) or (I) meet the following criteria:
Rn represent a hydrogen atom on all the carbon atoms of the carbon chain except one where Rn represents a linear or branched alkyl group comprising 1-6 carbon atoms, and being optionally substituted with one or more halogen atoms, in particular fluorine;
R represents a hydrogen atom or a linear or branched alkyl group comprising 1-6 carbon atoms, or a cycloalkyl group comprising 3-6 carbon atoms, and being optionally substituted with one or more halogen atoms, in particular fluorine; R preferably represents a hydrogen atom;
n, Ra, Rb and R₁ are as defined earlier.

According to a particular feature of the invention, the unsaturated fatty amino acid derivatives of general formula (A) or (I) meet the following criteria:
Ra and Rb represent a hydrogen atom.

According to a particular feature of the invention, the unsaturated fatty amino acid derivatives of general formula (A) or (I) meet the following criteria:
R represent a hydrogen atom.

According to a particular feature of the invention, the unsaturated fatty amino acid derivatives of general formula (A) or (I) meet the following criteria:
Rn represent a hydrogen atom.

According to a particular feature of the invention, the unsaturated fatty amino acid derivatives of general formula (A) or (I) meet the following criteria:
R₁ represents a hydrogen or fluorine atom.

According to a particular feature of the invention, the unsaturated fatty amino acid derivatives of general formula (A) or (I) meet the following criteria:
n is comprised between 3 and 5.

According to a particular feature of the invention, the unsaturated fatty amino acid derivatives of general formula (A) or (I) meet the following criteria:
n is comprised between 9 and 14.

According to a particular feature of the invention, the unsaturated fatty amino acid derivatives of general formula (A) or (I) meet the following criteria:
n is equal to 3.

The unsaturated fatty amino acid derivatives of general formula (A) or (I), as defined earlier, may exist as Z or E isomers or as a mixture of Z isomers and E isomers, in any amounts.

In the sense of the present invention, the term “pharmaceutically acceptable acid addition salts” means the salts formed by acid addition on the compound, which are non-toxic and which have the pharmacological activity of the parent compound.

The acid addition salts may be formed, from inorganic acids such as hydrochloric acid, hydrobromic acid, sulfuric acid, nitric acid or phosphoric acid, or else from organic acids such as acetic acid, benzene-sulfonic acid, benzoic acid, camphor-sulfonic acid, citric acid, ethane-sulfonic acid, fumaric acid, glucoheptonic acid, gluconic acid, glutamic acid, glycolic acid, hydroxynaphthoic acid, 2-hydroxyethane-sulfonic acid, lactic acid, maleic acid, malic acid, mandelic acid, methane-sulfonic acid, muconic acid, 2-naphthalene-sulfonic acid, propionic acid, salicylic acid, succinic acid, dibenzoyl-L-tartaric acid, tartaric acid, p-toluene-sulfonic acid, trimethylacetic acid or trifluoroacetic acid.

The preferred acceptable pharmaceutical salts are salts formed from hydrochloric acid.

The compounds, object of the present invention, preferably exist as hydrochloride salts.

Advantageously, the unsaturated fatty amino acid derivatives of the invention are selected from:
(E)-6-amino-hex-2-enoic acid hydrochloride,
(E)-7-amino-hept-2-enoic acid hydrochloride,
(E)-8-amino-oct-2-enoic acid hydrochloride,
(E)-9-amino-non-2-enoic acid hydrochloride,
(E)-10-amino-dec-2-enoic acid hydrochloride,
(E)-14-amino-tetradec-2-enoic acid hydrochloride,
6-amino-2-fluoro-hex-2-enoic acid hydro chloride as a mixture of Z and E isomers,
(Z)-6-amino-2-fluoro-hex-2-enoic acid hydrochloride,
(E)-6-amino-2-fluoro-hex-2-enoic acid hydrochloride,
7-amino-2-fluoro-hept-2-enoic acid hydrochloride as a mixture of isomers Z and E,
(Z)-7-amino-2-fluoro-hept-2-enoic acid hydrochloride,
9-amino-2-fluoro-non-2-enoic acid hydrochloride,
10-amino-2-fluoro-dec-2-enoic acid hydrochloride and
14-amino-2-fluoro-tetradec-2-enoic acid hydrochloride.

The present invention also relates to dermatological compositions comprising as an active ingredient at least one fatty amino acid derivative of general formula (A) or (I), as defined earlier for the derivatives as a drug, in association with an excipient acceptable in dermal cosmetology.

The present invention also relates to the use of unsaturated fatty amino acid derivatives of general formula (A) or (I), such as defined earlier for derivatives as a drug, for the preparation of an antiradical, anti-inflammatory, anti-pruriginous, anti-collagenase dermatological composition, and/or intended to treat skin ageing, and/or intended to treat disorders of keratinization and of pigmentation and/or intended to improve healing.

The unsaturated fatty amino acid derivatives of general formula (A) or (I) are more particularly intended for compositions intended to treat psoriasis, prurit, and/or atopic dermitis, as well as re-pigmentation of hair or of skin, notably of white age spots.

According to the invention, the unsaturated fatty amino acid derivatives of general formula (A) or (I) are more particularly intended for compositions intended to cause lightening of skin or treating brown age spots.

Because of their anti-radical activity, the unsaturated fatty amino acid derivatives of general formula (A) or (I) are also useful for avoiding or limiting cutaneous photocarcinogenesis at early stages and they therefore may be used in prevention and treatment of various tumoral diseases of the skin.

The present invention also relates to a method for preparing an unsaturated fatty amino acid derivative of the following general formula (A):

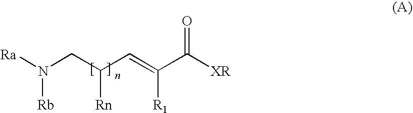

wherein:
X represents an oxygen or an NH group;
Rn represent independently of each other an hydrogen atom or a linear or branched alkyl group, comprising 1-6 carbon atoms, and being optionally substituted with one or more halogen atoms, in particular fluorine;
R₁ represents a hydrogen, fluorine, chlorine or bromine atom, or else a —CF₃ or —CHF₂ group or else a linear or branched alkynyl, alkenyl or alkyl group, comprising 1-10 carbon atoms, and being optionally substituted with one or more halogen atoms, in particular fluorine, and/or with one or more oxygen, nitrogen and/or sulfur atoms;
R represents a hydrogen atom or a linear or branched alkyl group, comprising 1-6 carbon atoms, or a cycloalkyl group comprising 1-6 carbon atoms, and being optionally substituted with one or more halogen atoms, in particular fluorine, and/or with one or more oxygen and/or nitrogen atoms;
Ra and Rb represent independently of each other a hydrogen atom or a linear or branched alkyl or acyl group comprising 1-6 carbon atoms and being optionally substituted with one or more oxygen and/or nitrogen atoms, Ra and Rb may form together a hydrocarbon cycle including 4-6 carbon atoms, optionally substituted with one or more oxygen and/or nitrogen atoms; and
n is an integer comprised between 2 and 14.
   said preparation method comprising:
the application of the Wittig-Horner reaction by reacting a phosphonate of the following formula (II):

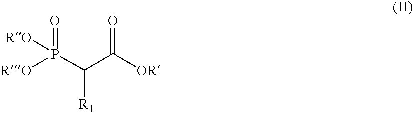

wherein:
R₁ is as defined above,
R′ represents a linear or branched alkyl group, comprising 1-6 carbon atoms, and preferably is an ethyl group, and
R″ and R″′ represent independently of each other, a linear or branched alkyl group, comprising 1-6 carbon atoms, and preferably a methyl or ethyl group, said groups R″ and R′″ being able to form an hydrocarbon cycle comprising 2-4 carbon atoms,
or, when R₁═H, the application of the Doebner-Knoevenagel reaction by reacting a malonic acid derivative of formula R′OOC—CH₂—COOR′, R′ being as defined above,
on a compound of the following formula (III):

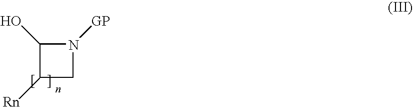

wherein:
   n is as defined above, and
   GP represents a protective group in order to notably form with the adjacent nitrogen atom a group of the carbamate type, N-GP being preferably a tert-butyl or benzyl carbamate,

GP notably corresponds to a protective group as defined in “Protective Groups in Organic Synthesis” Third edition Theodora GREEN & Peter WUTS Wiley Interscience ISBN 0-471-16019-9 Chapter 2 pages 17-246,

in order to obtain a compound of the following formula (IV):

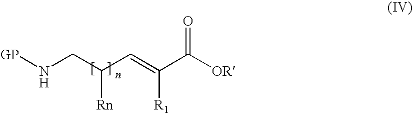

for which GP, R′, R₁, Rn and n are as defined above,
an optional saponification reaction of the compound of the aforementioned general formula (IV), in order to obtain a compound of the following general formula (V):

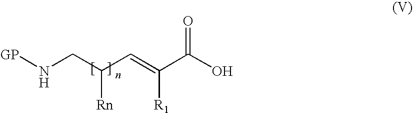

for which GP, R₁, Rn and n are as defined above,
a reaction for deprotecting the nitrogen of the compound of the aforementioned formula (IV) or (V), in order to obtain a compound of formula (A) wherein
Ra and Rb represent a hydrogen and X represents an oxygen,
and an optional N-alkylation and/or N-acylation and/or amidification reaction of the preceding compound of formula (A) wherein X represents an oxygen and Ra and Rb represent a hydrogen, in order to obtain a compound of formula (A) wherein at least one of the Ra and Rb groups does not represent a hydrogen.

The method of the present invention allows the preparation of unsaturated fatty amino acids, under excellent conditions both in terms of yield but also of quality without any trace of contamination, said method being transposable industrially.

The unsaturated fatty amino acid derivatives obtained with the method of the invention may exist as Z or E stereoisomers, or as a mixture of these different forms.

In a particular embodiment, the method of the invention allows the preparation of an unsaturated fatty amino acid of formula (I):

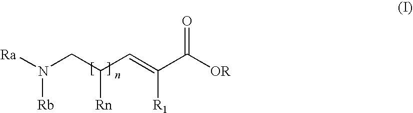

wherein:
Rn represent independently of each other a hydrogen atom or a linear or branched alkyl group comprising 1-6 carbon atoms, and being optionally substituted with one or more halogen atoms, in particular fluorine;
R₁ represents a hydrogen, fluorine, chlorine or bromine atom, or else a —CF₃ or —CHF₂ group or else a linear or branched alkynyl, alkenyl or alkyl group, comprising 1-6 carbon atoms, and being optionally substituted with a halogen atom, in particular fluorine;
R represents a hydrogen atom or a group R′ representing a linear or branched alkyl group comprising 1-6 carbon atoms, or a cycloalkyl group comprising 3-6 carbon atoms, and being optionally substituted with a halogen atom, in particular a fluorine atom;
Ra and Rb represent independently of each other a hydrogen atom or a linear or branched alkyl or acyl group comprising 1-6 carbon atoms, Ra and Rb may form together a hydrocarbon cycle including 4-6 carbon atoms; and
n is an integer comprised between 2 and 14,
   according to the different steps defined earlier for the preparation of the unsaturated fatty amino acid derivatives of formula (A).

In an advantageous way, the previous compound of general formula (III):

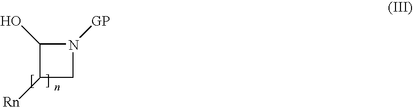

may be synthesized from a compound of general formula (VI):

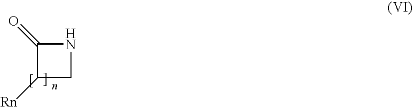

wherein n and Rn are as defined earlier,
according to the following steps:
protection of the nitrogen of the amide function of the compound of formula (VI) with a GP group in order to obtain a compound of the following general formula (VII):

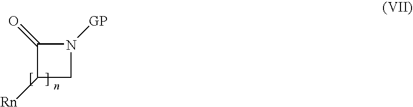

wherein n, Rn and GP are as defined earlier,
and then reduction of the carbonyl function of the compound of formula (VII) as defined earlier, this step being notably carried out with aluminium hydrides such as diisobutyl aluminium hydride at low temperature.

When Ra and/or Rb is different from a hydrogen atom, N-alkylation or N-acylation or amidification or a combination of these reactions are carried out according to methods known to one skilled in the art (“Mach's Advanced Organic Chemistry”, Fifth edition, ISBN O-471-58589-0, Michael. B. Smith and Jerry March, pages 501-552 and 511).

Unsaturated fatty amino acid derivatives of formula (A) or (I) wherein R is different from a hydrogen atom are conventionally obtained by coupling from the acid function.

If the compounds of structure (VI) are not available commercially, it is always possible to prepare them from the corresponding cyclic ketones according to methods well-known to one skilled in the art, i.e. by transformation of cyclic ketones into oximes by action of hydroxylamine, and then by treatment in a strong acid medium of these oximes into lactam according to a very standard Beckman reaction (“Mach's Advanced Organic Chemistry”, Fifth edition, ISBN O-471-58589-0, Michael. B. Smith and Jerry March, pages 1349, 1381, 1384, 1415-1416):

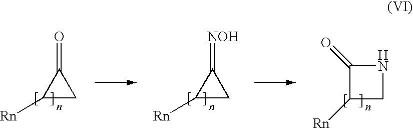

Also, when Rn is different from a hydrogen, it is possible to synthesize the compound (III) from the following hydroxyketone:

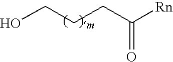

by the following chain reaction: protection of the alcohol function, Wittig-Horner reaction in order to obtain an α,β-unsaturated ester, hydrogenation of the double bond, reduction of the ester into an aldehyde, deprotection of the alcohol function and Mitsunobu or Gabriel reaction.

According to a particular embodiment of the method of the invention, Ra and Rb represent hydrogens.

According to a particular embodiment of the method of the invention, R represents a hydrogen.

According to still another particular embodiment of the method of the invention, Rn represent a hydrogen.

According to yet another particular embodiment of the method of the invention, R₁ represent a hydrogen or a fluorine.

The present invention also relates to a method for preparing an unsaturated fatty amino acid derivative of the following formula (1-I):

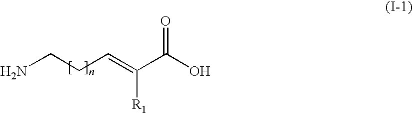

corresponding to a compound of general formula (I) wherein:
Ra, Rb, Rn and R represent a hydrogen; and
R₁ is as defined earlier, i.e. it represents a hydrogen, fluorine, chlorine or bromine atom, or else a —CF₃ or —CHF₂ group or else a linear or branched alkynyl, alkenyl or alkyl group, comprising 1-6 carbon atoms, and being optionally substituted with a halogen atom, in particular fluorine, and/or with one or more oxygen, nitrogen and/or sulfur atoms; and
n represents an integer comprised between 2 and 14,
   said preparation method comprises the following steps:
a step for protecting the amide function of a compound of the following formula (VI-1), which corresponds to a compound of formula (VI) for which Rn=H:

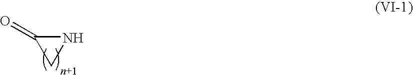

wherein n is as defined above in formula (I-1),
in order to obtain a compound of the following formula (VII-I), in which the amide function is protected, and corresponding to a compound of formula (VII) for which Rn=H:

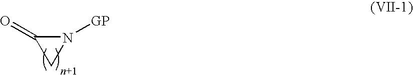

wherein:
n is as defined above in formula (I-1), and
GP represents a protective group in order to form with the adjacent nitrogen atom notably a group of the carbamate type, N-GP preferably being a t-butyl or benzyl carbamate,

GP corresponding to a protective group as defined in “Protective Groups in Organic Synthesis” Third edition Theodora GREEN & Peter WUTS Wiley Interscience ISBN 0-471-16019-9 Chapter 7 pages 494-654,
a step for reducing the carbonyl function of the compound of formula (VII-1) as defined above in order to obtain a compound of the following formula (III-1), corresponding to a compound of formula (III) for which Rn=H:

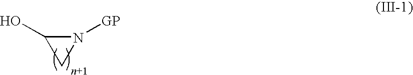

n and GP being as defined above in formula (VII-1),

this step being notably carried out with conventional reducing agents such as hindered aluminium hydrides such as diisobutyl aluminium hydride at low temperature,

the application of the Wittig-Horner reaction by reacting a phosphonate of the following formula (II)

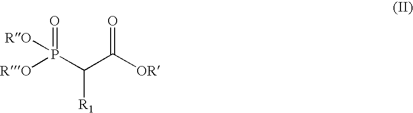

wherein:
R₁ is as defined above,
R′ represents a linear or branched alkyl group comprising 1-6 carbon atoms and preferably is an ethyl group, and
R″ and R″′ represent independently of each other a linear or branched alkyl group comprising 1-6 carbon atoms and preferably an ethyl or methyl group, said groups R″ and R″′ being able to form together a hydrocarbon cycle comprising 2-4 carbon atoms,

or, when R₁═H, the application of the Doebner-Knoevenagel reaction by reaction of a malonic acid derivative of formula R′OOC—CH₂—COOR′, R′ being as defined above,

on the aforementioned protected compound of formula (III-1),

in order to obtain a compound of the following formula (IV-1), corresponding to a compound of formula (IV) for which Rn=H:

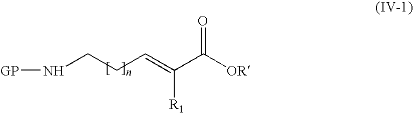

wherein n, GP, R₁ and R′ are as defined above.

a saponification reaction of the compound of the aforementioned formula (IV-1), in order to obtain a compound of the following formula (V-1), corresponding to a compound of general formula (V) for which Rn=H:

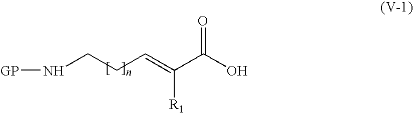

wherein n, GP and R₁ are as defined above,

and a reaction for deprotecting the nitrogen of the compound of the aforementioned formula (V-1) in order to obtain a compound of formula (I-1).

The unsaturated fatty amino acids, object of the present invention, may thus be prepared by applying reactions of the Wittig-Horner type from a phosphonate (either fluorinated or not) or of the Wittig type starting with a triphenylphosphonium on the cyclized alpha hydroxylamine derived from the corresponding lactam, the amine function of which will be protected beforehand (see the synthesis scheme developed hereafter).

Possibly, a saponification reaction of the protected amino ester follows in order to obtain an amino acid which will be deprotected by acid hydrolysis in order to obtain the unsaturated fatty amino acid of general formula:

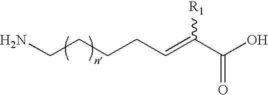

n′=n−2 and R₁═H, halogen or alkyl

The Wittig-Horner reaction is a reaction described in the document Modern Synthetic Reaction, Second edition, Herbet O. House, Wittig-Horner reaction p. 682-709, and any experimental condition described in the state of the art may be used within the scope of the present invention. As an example, the Wittig-Horner reaction may be carried out in the presence of triethylphosphonoacetate and of potassium carbonate in an ethanol medium.

The method for protecting the amine function is a standard method, commonly used for its advantage of not being hydrolysable under basic conditions (conditions for deprotecting the ester function) and inert towards other nucleophilic reagents (References: T. Kunieda, T. Higuchi, Y. Abe, and M. Hirobe, Chem. Pharm. Bull., 32, 2174, 1984; I. Grapsas, Y. J. Cho, and S. Mobashery, J. Org. Chem. 59, 1918; 1994).

The step for partly reducing the lactam is carried out in the presence of a reducing agent such as diisobutyl aluminium hydride. At the end of the reaction, the aluminium salts are removed by forming a water-soluble complex in the presence of Rozen salts (Reagents for organic synthesis, Vol. 1, Louis Fieser and Mary Fieser, p. 36).

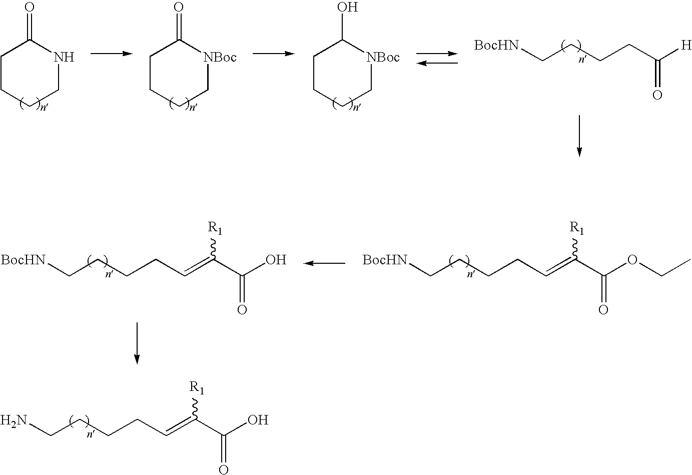

An example for synthesizing a derivative wherein Rn=methyl may be illustrated by a chain of protection, deprotection, Wittig-Horner and Gabriel reactions starting with a hydroxy ketone. After protecting the alcohol function, a succession of Wittig-Horner reactions is carried out. The deprotection of the alcohol function in an acid medium, followed by a Gabriel reaction, will generate the amine function.

The present invention also relates to compounds, as synthesis intermediates, of the following general formula (VIII):

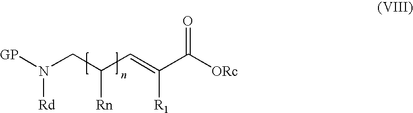

for which:
Rc represents a hydrogen or a linear or branched alkyl group comprising 1-6 carbon atoms,
R₁ represents a hydrogen, fluorine, chlorine or bromine atom, or else a —CF₃ or —CHF₂ group or else a linear or branched alkynyl, alkenyl or alkyl group comprising 1-6 carbon atoms, and being optionally substituted with one or more halogen, in particular fluorine, oxygen, nitrogen and/or sulfur atoms;
Rn represent independently of each other a hydrogen atom or a linear or branched alkyl group comprising 1-6 carbon atoms, and being optionally substituted with one or more halogen atoms, in particular fluorine;
n is an integer comprised between 2 and 14.
GP represents a protective group in order to notably form with the adjacent nitrogen atom a group of the carbamate type, N-GP being preferably a tert-butyl or benzyl carbamate,

GP notably corresponds to a protective group as defined in “Protective Groups in Organic Synthesis” Third edition Theodora GREEN & Peter WUTS Wiley Interscience ISBN 0-471-16019-9 Chapter 2 pages 17-246; and
Rd represents a hydrogen or a protective group GP′ as defined above for GP, and in particular such that N-GP′ represents a tert-butyl or benzyl carbamate;

the following compounds being excluded: tert-butyl (E)-6(N,N-di-tert-butoxy-carbonylamino)hex-2-enoate, ethyl(E)-6(N-tert-butoxy-carbonylamino)hex-2-enoate, methyl(E)-7-(N-tert-butoxy-carbonylamino)hept-2-enoate and (E)-7(N-tert-butoxy-carbonylamino)hept-2-enoic acid.

According to a particular characteristic of the invention, the compounds of formula (VIII) meet the following criterion: R₁ represents a hydrogen, fluorine, chlorine, or bromine atom, or else a-CF₃ or —CHF₂ group or else a linear or branched alkynyl, alkenyl or alkyl group, comprising 1-6 carbon atoms, and being optionally substituted with one or more halogen atoms, in particular fluorine.

According to a particular feature of the invention, the compounds of formula (VIII) meet the following criterion: Rd represents a hydrogen.

According to a particular feature of the invention, the compounds of formula (VIII) meet the following criterion: Rn represent a hydrogen.

According to another particular feature of the invention, the compounds of formula (VIII) meet the following criterion: R₁ represents a hydrogen or a fluorine.

The present invention will be illustrated within the scope of the synthesis examples given hereafter:

### 1) SYNTHESIS OF THE COMPOUNDS ACCORDING TO THE INVENTION

### 1.1) Compound No. 12 ((E)-10-amino-dec-2-enoic acid)

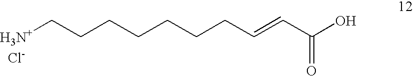

### Stage 1

In a three-neck flask, under a flow of nitrogen, 10.0 g of caprylolactam (70.8 mmol) are solubilized in 150 mL of tetrahydrofurane. 10.06 mL (1.01 eq.) of triethylamine are added as well as 8.74 g (1.01 eq.) of 4-dimethylaminopyridine, and finally 30.91 g (2 eq.) of di-tert-butyl di-carbonate. The reaction medium is stirred overnight at room temperature. By tracking with TLC, it is possible to check for the end of the reaction. The reaction medium is concentrated, taken up with water and ethyl acetate. The mixture is extracted three times with ethyl acetate, the collected organic phases are washed with a 5% hydrochloric acid solution, and then with a saturated NaCl solution. The organic phases are dried on Na₂SO₄, filtered and concentrated in vacuo, in order to lead to a red oil.

### Characterizations:

m=24.0 g 100% yield

R_{f}=0.8 (DCM/MeOH 99/1)

NMR (¹H, CDCl₃): 1.50 (s, 9H); 1.53 (m, 6H); 1.67 (m, 2H); 1.82 (m, 2H); 2.87 (m, 2H), 2.78 (t, 2H).

### Stage 2

In a three-neck flask, under a flow of nitrogen, 17.1 g of caprylolactam protected by a Boc group (70.8 mmol) are solubilized in 170 mL of toluene. The medium is cooled down to −78° C. and 59.2 mL of a 20% Dibal-H solution in toluene (1.01 eq) are poured dropwise, over 1 hour, while maintaining the temperature of the medium between −80° C. and −75° C. By tracking with TLC, it is possible to check for the end of the reaction. At −78° C., 480 mL of a saturated double tartrate solution are poured slowly and the mixture is vigorously stirred overnight. The organic phase is extracted. The aqueous phase is extracted three times with ethyl acetate. The collected organic phases are washed with a saturated NaCl solution, dried on MgSO₄, filtered and concentrated in vacuo, in order to lead to an orange solid

### Characterizations:

m=20.0 g 99% yield

R_{f}=0.1 (heptane/ethyl acetate 7/3)

NMR (¹H, CDCl₃): 1.40-1.80 (m, 20H); 2.85-2.89 (m, 2H); 3.75-3.79 (m, 2H).

### Stage 3

In a three-neck flask, under a flow of nitrogen, 2.0 g (8.22 mmol) of compound from stage 2 are solubilized with 20 mL of ethanol. 1.70 (1.5 eq) of potassium carbonate are added to the medium and 1.96 mL (1.2 eq) of triethylphosphonoacetate are slowly poured. The mixture is then heated to 45° C. overnight. By tracking with TLC, it is possible to check for the end of the reaction. The reaction medium is concentrated and then the residue is taken up with ethyl acetate and water. The aqueous phase is extracted with ethyl acetate three times. The collected organic phases are washed with a saturated NaCl solution, dried on MgSO₄, filtered and concentrated in vacuo. The obtained crude product is purified by chromatography on silica gel (heptane/AcOEt gradient) leading to a yellow oil.

### Characterizations:

m=1.60 g 62% yield

R_{f}=0.4 (heptane/ethyl acetate 7/3)

NMR (¹H, CDCl₃, 300 MHz): δ 1.28-1.46 (m, 22H); 2.16-2.19 (m, 2H); 3.10 (m, 2H); 4.20 (q, 2H); 5.82 (dt, 1H, J=15.6 Hz), 6.97 (dt, 1H, J=15.6 Hz).

### Stage 4

In a flask, 1.60 (5.10 mmol) of the compound from stage 3 are put into solution in 16 mL of THF. 6.40 mL (2.5 eq) of a 2M NaOH solution are added and the reaction medium is heated to 65° C. overnight. By tracking with TLC, it is possible to check for the end of the reaction. The pH of the mixture is then adjusted to 4 by adding hydrochloric acid. The medium is concentrated, taken up with AcOEt/water. The aqueous phase is extracted with ethyl acetate (three times); the collected organic phases are washed with a saturated NaCl solution, dried on Na₂SO₄, filtered and concentrated in vacuo, leading to a beige solid.

### Characterizations:

m=1.2 g 82% yield

R_{f}=0.2 (heptane/ethyl acetate 7/3)

NMR (¹H, CDCl₃, 300 MHz); δ 1.30-1.46 (m, 19H); 2.20-2.27 (m, 2H); 3.13 (m, 2H); 5.80.5.86 (dt, 1H, J=15.6 Hz); 7.0 (dt, 1H, J=15.6 Hz).

### Stage 5

In a flask under a balloon of nitrogen, 1.2 g (4.28 mmol) of compound from stage 4 are solubilized in 7.5 mL (3.5 eq) of a 2M HCl solution in ether. The medium is stirred overnight at 35° C. By tracking with TLC, it is possible to check for the end of the reaction. The medium is concentrated in vacuo, taken up in dichloromethane, filtered and washed with dichloromethane, dried in vacuo, leading to a white solid. This solid is recrystallized several times from ethanol.

### Characterizations:

m=0.59 g 82% yield

R_{f}=0.1 (heptane/ethyl acetate 5/5)

NMR (1H, MeOD, 300 MHz); δ 1.50-1.69 (m, 10H); 2.21-2.28 (q, 2H); 2.93 (t, 2H); 5.80 (d, 1H, J=15.6 Hz); 6.95 (dt, 1H, J=15.6 Hz).

Mass spectrometry: [M+H]⁺=186 (calculated 185).

### 1.2) Compound No. 13 (10-amino-2-fluoro-dec-2-enoic acid)

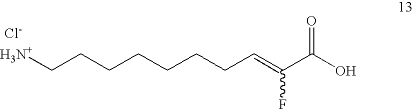

Stages 1 and 2 are conducted in the same way as earlier on caprylolactam.

### Stage 3

In a three-neck flask, under a flow of nitrogen, 2.0 g (8.22 mmol) of compound from stage 2 are solubilized with 20 mL of ethanol. 1.70 g (1.5 eq) of potassium carbonate are added to the medium and 2.06 mL (1.1 eq) of triethylphosphonofluoroacetate are slowly poured. The mixture is then heated to 45° C. overnight. By tracking with TLC, it is possible to check for the end of the reaction. The reaction medium is concentrated and then the residue is taken up with ethyl acetate and water. The aqueous phase is extracted with ethyl acetate three times. The collected organic phases are washed with a saturated NaCl solution, dried on MgSO₄, filtered and concentrated in vacuo. The obtained crude product is purified by chromatography on silica gel (heptane/AcOEt gradient) leading to a pale yellow oil.

### Characterizations:

m=1.70 g 65% yield

R_{f}=0.5 (heptane/ethyl acetate 7/3)

NMR (¹H, CDCl₃, 300 MHz); δ 1.32-1.39 (m, 22H); 2.21 (m, 2H); 3.11 (m, 2H); 4.30 (q, 2H); 5.89-5.96 (dt, 0.5H, J=21.9 Hz, form E); 6.06-6.18 (dt, 0.5H, J=33.3 Hz, form Z).

Stages 4 and 5 are conducted as earlier and lead to the following characterizations:

### Characterizations of Stage 4:

m=1.4 g 87% yield

R_{f}=0.2 (heptane/ethyl acetate 7/3)

NMR (¹H, CDCl₃); δ 1.23-1.46 (m, 19H); 2.26-2.55 (m, 2H); 3.12 (m, 2H); 5.98-6.32 (ddt, 1H, form E and form Z).

### Characterizations of Stage 5:

m=0.9 g 95% yield

R_{f}=0.1 (heptane/ethyl acetate 5/5)

NMR (41, MeOD); δ 1.41-1.53 (m, 8H); 1.65-1.70 (m, 2H), 2.24-2.29 (m, 1H form Z); 2.53-2.56 (m, 1H, form E); 2.91-2.96 (t, 2H), 5.90-6.03 (dt, 0.5H, J=21.6 Hz, form E); 6.08-6.25 (dt, 0.5H, J=33.3 Hz, form Z).

Mass spectrometry: [M+H]⁺=204 (calculated 203)

### 1.3) Compound No. 7 ((E)-8-amino-oct-2-enoic acid)

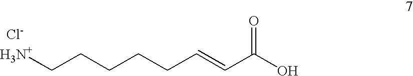

The general procedure used for preparing the compound 12 is applied to s-caprolactam, in order to lead to (E)-8-amino-oct-2-enoic acid.

R_{f}=0.1 (heptane/ethyl acetate 5/5)

NMR (¹H, MeOD); δ 1.41-1.57 (m, 4H); 1.67-1.75 (m, 2H); 2.24-2.31 (m, 2H); 2.93-2.98 (m, 2H); 5.86 (dd, 1H); 6.98 (dt, 1H).

Mass spectrometry: [M+H]⁺=158 (calculated 157)

### 1.4) Compound No. 8 ((E)-9-amino-non-2-enoic acid)

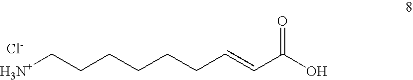

The general procedure used for preparing the compound 12 is applied to oenantholactam in order to lead to (E)-9-amino-non-2-enoic acid.

R_{f}=0.1 (heptane/ethyl acetate 5/5)

NMR (¹H, DMSO-d₆); δ 1.29-1.54 (m, 8H); 2.16-2.23 (m, 2H); 2.71-2.76 (m, 2H); 5.77 (dd, 1H, J=18 Hz); 6.81 (dt, 1H, J=18 Hz).

Mass spectrometry: [M+H]⁺=172 (calculated 171)

Melting point: 104° C.

### 1.5) Compound No. 9 (9-amino-2-fluoro-non-2-enoic acid)

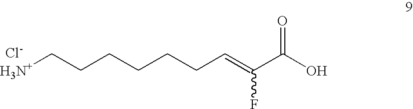

The general procedure used for preparing the compound 13 is applied to oenantholactam in order to lead to 9-amino-2-fluoro-non-2-enoic acid.

R_{f}=0.1 (heptane/ethyl acetate 5/5)

NMR (¹H, MeOD); δ 1.40-1.71 (m, 8H); 2.30-2.35 (m, 2H); 2.91-2.96 (m, 2H); 5.92-6.05 (dt, 0.5H, J=21.9 Hz, form E); 6.09-6.26 (dt, 0.5H, J=33.3 Hz, form Z).

Mass spectrometry: [M−H]⁻=188 (calculated 189)

Melting point: 120° C.

### 1.6) Compound No. 11 ((E)-14-amino-tetradec-2-enoic acid)

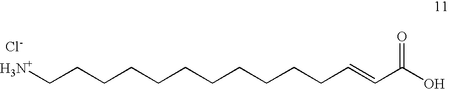

The general procedure used for preparing the compound 12 is applied to azacyclodecanone in order to lead to (E)-14-amino-tetradec-2-enoic acid.

R_{f}=0.1 (heptane/ethyl acetate 5/5)

NMR (¹H, MeOD); δ 1.31-1.69 (m, 18H); 2.20-2.27 (m, 2H); 2.90-2.95 (m, 2H); 5.80 (dd, 1H, J=15.6 Hz); 6.96 (dt, 1H, J=15.6 Hz).

Mass spectrometry: [M+H]⁺=242 (calculated 241)

Melting point: 153° C.

### 1.7) Compound No. 10 (14-amino-2-fluoro-tetradec-2-enoic acid)

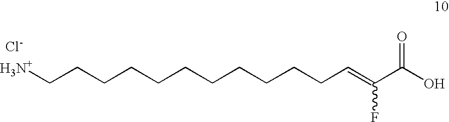

The general procedure used for preparing the compound 13 is applied to azacyclodecanone in order to lead to 14-amino-2-fluoro-tetradec-2-enoic acid.

R_{f}=0.1 (heptane/ethyl acetate 5/5)

NMR (¹H, MeOD); δ 1.34-1.67 (m 18H); 2.21-226 (m, 1H form Z); 2.48-2.54 (m, 1H, form E); 2.92 (t, 2H), 5.91-6.04 (dt, 0.5H, J=21.9 Hz, form E); 6.09-6.25 (dt, 0.5H, J=33.3 Hz, form Z).

Mass spectrometry: [M+H]⁺=260 (calculated 259)

Melting point: 148.5° C.

### 1.8) Compound No. 1 ((E)-6-amino-hex-2-enoic acid)

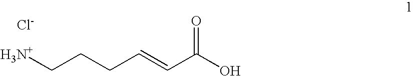

The general procedure used for preparing the compound 12 is applied to 2-pyrrolidone in order to lead to (E)-6-amino-hex-2-enoic acid.

R_{f}=0.1 (heptane/ethyl acetate 5/5)

NMR (¹H, MeOD); δ 1.81-1.91 (m, 2H); 2.32-2.40 (m, 2H); 2.95-3.00 (m, 2H); 5.90 (dd, 1H, J=15.6 Hz); 6.96 (dt, 1H, J=15.6 Hz).

Mass spectrometry: [M+H]⁺=130 (calculated 129)

### 1.9) Compound No. 2 ((E)-6-amino-2-fluoro-hex-2-enoic acid) and compound No. 3 ((Z)-6-amino-2-fluoro-hex-2-enoic acid)

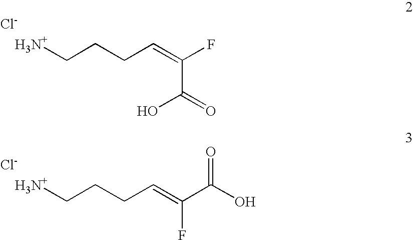

The general procedure used for preparing the compound 13 is applied to 2-pyrrolidone in order to lead to (Z)-6-amino-2-fluoro-hex-2-enoic acid (No. 3) and to (E)-6-amino-2-fluoro-hex-2-enoic acid (No. 2), which were separated in this case.

### Form Z

R_{f}=0.1 (heptane/ethyl acetate 5/5)

NMR (¹H, MeOD); δ 1.80-1.90 (m, 2H); 2.33-2.42 (m, 2H); 2.95-300 (m, 2H); 6.12-6.28 (dt, 1H, J=32.7 Hz, form Z).

Mass spectrometry: [M+H]⁺=148 (calculated 147)

### Form E

R_{f}=0.1 (heptane/ethyl acetate 5/5)

NMR (¹H, MeOD); δ 1.77-1.89 (m 2H); 2.60-2.68 (m, 2H); 2.94-2.99 (m, 2H); 5.96-6.08 (dt, 1H, J=21.0 Hz, form E).

Mass spectrometry: [M+H]⁺=148 (calculated 147)

### 1.10) Compound No. 4 ((E)-7-amino-hept-2-enoic acid

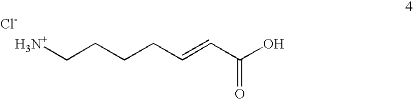

The general procedure used for preparing the compound 12 is applied to δ-valerolactone in order to lead to (E)-7-amino-hept-2-enoic acid.

R_{f}=0.1 (heptane/ethyl acetate 5/5)

NMR (¹H, MeOD); δ 1.56-1.74 (m, 4H); 2.28-2.34 (m, 2H); 2.94-2.99 (m 2H); 5.84 (dd, 1H, J=15.6 Hz); 6.97 (dt, 1H, J=15.6 Hz).

Mass spectrometry: [M+H]⁺=144 (calculated 143)

### 1.11) Compound No. 5 ((Z+E)-7-amino-2-fluoro-hept-2-enoic acid) and compound No. 6 ((Z)-7-amino-2-fluoro-hept-2-enoic acid)

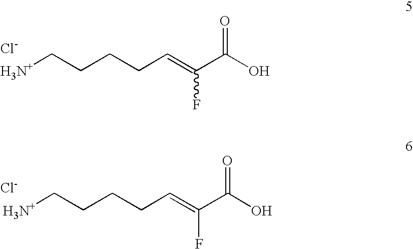

The general procedure used for preparing the compound 13 is applied to δ-valerolactone in order to lead to (Z)-7-amino-2-fluororo-hept-2-enoic acid (No. 6) and to the mixture (Z+E)-7-amino-2-fluoro-hept-2-enoic acid (No. 5).

### Form Z

R_{f}=0.1 (heptane/ethyl acetate 5/5)

NMR (¹H, MeOD); δ 1.55-1.71 (m, 4H); 2.32-2.35 (m, 2H); 2.93-2.98 (m, 2H); 6.11-6.28 (dt, 1H, J=33.3 Hz, form Z).

Mass spectrometry: [M+H]⁺=162 (calculated 161)

### Form E+Z

R_{f}=0.1 (heptane/ethyl acetate 5/5)

NMR (¹H, MeOD); δ 1.54-1.75 (m 4H); 2.30-2.41 (m, 0.6H); 2.56-2.64 (m, 1.4H, form E); 2.94-2.99 (m 2H); 5.95-6.08 (dt, 0.7H, J=21.6 Hz, form E); 6.12-6.28 (dt, 0.3H, J=33.3 Hz, form Z).

Mass spectrometry: [M+H]⁺=162 (calculated 161).

### 2) ANTI-INFLAMMATORY EFFECT ANALYZED AT THE LIPID MEDIATORS OF THE INFLAMMATION

The keratinocyte, the most represented cell at the epidermis, releases, in response to many extracellular factors present in its environment, biologically active mediators, notably prostaglandins and leukotrienes which play an important role in initiating and modulating cutaneous inflammatory reactions and which are also involved in regulating the immune response. The prostaglandin PG6KF1 alpha, is one of the major metabolites produced by the stimulated keratinocyte, and representative of the modulation of the production of metabolites of the metabolism of arachidonic acid stemming from the cyclo-oxygenase route.

### 2.1) Procedure

The suspension of keratinocytes in DMEM with 10% FCS is distributed in plates of 6 wells (1.2 106 cells/well), and incubated for 16 hours at 37° C. in an atmosphere with 5% CO₂. The keratinocytes are then rinsed with PBS in order to eliminate non-adherent cells and then exposed to the products to be tested included in DMEM without FCS (which might interfere in the assay).

The tested culture concentration is 3 μg/mL. It was retained after a preliminary test for evaluating cytotoxicity (neutral red) and it is not cytotoxic.

For each treatment, 3 wells were produced. The cells were pre-incubated for 60 min with the products to be tested and then an agent stimulating the cascade of arachidonic acid, the calcium ionophore, is added for 5 hours: the calcium ionophore A23187 is used at a concentration of 1 μM.

After these 5 hours of culture, the culture media of each of the wells are recovered, centrifuged at 3,000 rpm and stored at −80° C.

The production of prostaglandin 6KF1α for each of the tests is measured with the Elisa kit EUROMEDEX.

### 2.2) Results

The results are gathered in Table 1 and are expressed in percentage of activity/stimulated control.

TABLE 1

| | | | | | | Inhibitory activity % of |
|---|---|---|---|---|---|---|
| Compounds of formula I | | | | | | molecules evaluated on the |
| No. | n | R | Rₙ | R₁ | Salt | production of PG6KF1α |
| 1 | 2 | H | H | H | HCl | + |
| 2 | 2 | H | H | F | HCl | ~ |
| 3 | 2 | H | H | F | HCl | 22 |
| 4 | 3 | H | H | H | HCl | 18 |
| 5 | 3 | H | H | F | HCl | 41 |
| 6 | 3 | H | H | F | HCl | 25 |
| 7 | 4 | H | H | H | HCl | 36 |
| 8 | 5 | H | H | H | HCl | 8 |
| 9 | 5 | H | H | F | HCl | 17 |
| 10 | 10 | H | H | F | HCl | 38 |
| 11 | 10 | H | H | H | HCl | 40 |

The synthesis of the obtained data allows demonstration of the anti-inflammatory potentialities in particular of compounds Nos. 11, 5, 7 and 10, with an effective dose 45 (DE45) for the compound No. 11 of 10 μg/mL and 25 μg/mL for the compound No. 5.

### 3) ANTI-INFLAMMATORY PROPERTY

### Inhibition of the Synthesis of a Pro-Inflammatory Cytokine IL8

With the skin barrier function, it is possible to ensure protection against the external environment, and the keratinocytes of the epidermis may directly respond to a large variety of irritating or allergenic agents and actively participate in cutaneous inflammatory and immune processes, in particular via the generation of pro-inflammatory cytokines, mediators of protein origin. Among these biologically active molecules, IL1α (Interleukin 1α) and TNF α (Tumor Necrosis Factor α) are considered as primary cytokines, their release being sufficient for inducing inflammation by virtue of their induction of adhesion molecules at the endothelial cells and of their induction of chimiotactic factors such as chemokines. The chemokine system controls leukocyte traffic during the inflammatory response and is required for interactions of the innate and adaptive immune responses.

In the present study, we were more specifically interested in the chemokine—Interleukin 8—which is strongly involved in amplifying the inflammatory response and the main function of which is to recruit and activate neutrophilic polynuclears notably by stimulating their release of pro-inflammatory molecules.

In this study conducted on 96-well plates, we evaluated the activity of amino-alkene acids, on the production of interleukin 8 induced at the keratinocyte by the phorbol ester PMA and the calcium ionophore A23187.

### 3.1) Procedure

The suspension of keratinocytes in supplemented KSFM is distributed into plates of 96 wells (3.10⁴ cells/well), and incubated for 16 hours at 37° C. in an atmosphere with 5% CO₂. The keratinocytes are then rinsed with PBS in order to remove the non-adherent cells and then exposed to the products to be tested included in non-supplemented KSFM (which might interfere in the assay).

The tested culture concentration is 3 μg/mL. It was retained after a preliminary test for evaluating cytotoxicity (neutral red) and it is not cytotoxic.

For each treatment, 3 wells are produced. The cells are pre-incubated for 60 min with the products to be tested and then stimulated, in parallel with negative controls without any stimulant: Phorbol Myristate Acetate (PMA) 1 μM+calcium Ionophore (A23187) 0.1 μM.

Incubation for 6 hours at 37° C. in a humid air atmosphere containing 5% of CO₂.

The culture media of each of the wells are recovered, centrifuged at 3,000 rpm and stored at −80° C.

Assay of the cytokines: IL8 is assayed by an immunoenzymatic method with an ELISA kit (Immunotech).

### 3.2) Results

The results are gathered in Table 2 and are expressed in percentage of activity/stimulated control.

TABLE 2

| | | | | | | Inhibitory activity % of |
|---|---|---|---|---|---|---|
| Compounds of formula I | | | | | | molecules evaluated on the |
| No. | n | R | Rₙ | R₁ | Salt | production of IL8 |
| 1 | 2 | H | H | H | HCl | ~ |
| 2 | 2 | H | H | F | HCl | ~ |
| 3 | 2 | H | H | F | HCl | ~ |
| 4 | 3 | H | H | H | HCl | ~ |
| 5 | 3 | H | H | F | HCl | + |
| 6 | 3 | H | H | F | HCl | + |
| 7 | 4 | H | H | H | HCl | ~ |
| 8 | 5 | H | H | H | HCl | 14 |
| 9 | 5 | H | H | F | HCl | 20 |
| 10 | 10 | H | H | F | HCl | 8 |
| 11 | 10 | H | H | H | HCl | 22 |

With the summary of the obtained data, the anti-inflammatory potentialities in particular of compounds Nos. 11 and 9, with an effective dose 50 (DE50) of 11 μg/mL for compound No. 11, may be demonstrated.

### 4) STUDY OF THE ANTI-RADICAL EFFECT ON OXYGEN ACTIVE SPECIES (OAS)

During normal cellular metabolism, during an occasional exposure of the skin to stressing agents or during dermatological pathologies, reactive oxygenated species further called Oxygen Activated Species (OAS) are generated (Y. M. W. Jansen et al, 1993). These OASes, described as very reactive metabolites, play an important role in a good number of processes such as inflammation, ageing and tumoral promotion.

The OASes are considered as “second messengers” in the cellular signaling of oxidative stress and therefore as early mediators of the inflammation (A. Van Der Vliet and A. Bast, 1992).

Their excess production induces significant damages in the cell. Certain cell constituents are then the major targets of such an oxidative stress: the lipid components of the plasma membrane (lipoperoxidation), the proteins (denaturation and degradation) and the genetic material or DNA (mutations) may be altered. The cells are capable of limiting these oxidative damages by means of different antiradical defense systems (enzymatic and non-enzymatic antioxidants) (B. P. Yu. 1994; H. Steiling et al, 1999).

However, under certain conditions, the OASes are produced in an amount such that cellular antioxidant activity is insufficient; these OASes then become factors inducing inflammatory pathologies and tissue aging (Y. Miyachi et al, 1986; MK Kress et al, 1995).

There are different chemical agents (e.g.: H₂O₂) or physical agents (eg.: WA) capable of generating an oxidative stress in vitro. Thus the produced OASes will alter various cell targets (membranes, DNA or proteins), the alteration of which may be analyzed by biochemical methodologies widely used such as assaying TBARS for lipid lipoperoxidation, or in vitro assaying the intracellular OASes by means of the probe H₂DCF-DA.

We have set up a model for an in vitro study of oxidative stress induced by H₂O₂/iron, H₂O₂ which massively generates intracellular OASes by a chain reaction triggered by the oxidation of membrane lipids.

This technique is based on the use of a fluorescent probe, 6-carboxy-2′, 7′ dichlorodihydrofluorescein diacetate, di(acetoxymethyl ester) (H₂DCF-DA), which, once it has penetrated the cell, is deacetylated by the intracellular esterases then forming H₂DCF. This product is oxidized by intracellular OASes into a highly fluorescent compound: 2′,7′-dichlorofluorescein, (DCF) (Suematsu M et al, 1996, Free Radicals Practical Approach, Punchard ed. P83-99).

The equipment and methods used for in vitro assaying intracellular OASes are indicated hereafter.

a) Cellular Tool:

Cutaneous murine fibroblast cell line L929.

b) Equipment:
96-well microplate with flat bottom
Cytofluorimeter Cytofluor II: Ref. PERSEPTIVE BIOSYSTEMS

c) Reagents:

### Cell Culture Reagents:

Culture medium: Dulbecco's Modified Eagle Medium (DMEM)
Fetal calf serum (FCS)
Phosphate buffer PBS ph 7.4
Trypsin EDTA (1×)

### Fluorescent Probe:

6-carboxy-2′,7′-dichlorodihydrofluorescein diacetate, di(acetoxymethyl ester) (MW: 675.43)

### Cell Stimulation Products:

hydrogen peroxide (H₂O₂) 3%: Ref. GIFRER (Laboratoire Gifrer Barbezat)
Ferrous ammonium sulfate (Fe²⁺)
Ferric ammonium sulfate (Fe^{3÷})

d) Tested Products:

The tested concentrations are non-cytotoxic concentrations. Cytoxicity was evaluated by the neutral red method, after incubation of the product for 3 hours.

The reference anti-radical product is vitamin E or α-tocopherol (MW: 430.7) (SIGMA, ref.: T-1539).

The mother solution is prepared at 400 mg/mL in DMSO and stored at −20° C. The pretreatment solution is prepared extemporaneously at 400 μg/mL in a culture medium without FCS.

For evaluating unsaturated fatty amino acid derivatives, the dilutions are prepared in the culture medium extemporaneously for a range of concentrations of 0.02, 0.2, 2 and 20 ng/mL.

e) Procedure:

### Sowing the Cells:

The cells of the fibroblast line L929 are sown in microplates with 96 wells with a flat bottom in 100 μL of DMEM supplemented with 10% of FCS, and they are then incubated overnight at 37° C. in a humid atmosphere with 5% CO₂.

The plate blank without cells is evaluated on 6 wells.

### Pre-Treatment of the Cells:

The dilutions of the products to be tested and of the reference molecule are conducted in culture mediums without FCS, and then deposited in 7 wells in an amount of 100 μL per well.

The cells are then incubated for 3 hours at 37° C. in a humid atmosphere with 5% CO₂.

The “control” cells (natural cell fluorescence), “probed control” cells (basal production of OAS) and “stimulated” cells (production of OAS after oxidative treatment) are covered with 100 μM of DMEM.

The “probed control” cells are incubated with the probe but are neither pretreated nor treated.

The “stimulated” cells are incubated with the probe and are treated but not pretreated.

The “control” cells are neither pretreated nor incubated with the probe nor treated.

Incubation of the Cells with the Probe and Oxidative Stress:

The cells are rinsed with PBS 1× in an amount of 100 μL per well. Next they are set to incubate for 30 min at 37° C. in a humid atmosphere with 5% CO₂ and with 50 μL of H₂DCF-DA probe at 5 μM.

After 30 min in contact with the probe alone, the cells are incubated for 30 min at 37° C. in a humid atmosphere with 5% O₂ with addition of 25 μL of H₂O₂ at 800 μM and 25 μL of 8 mM ferrous and ferric iron solution, in order to have final concentrations of 200 μM of H₂O₂ and 2 mM of ferrous and ferric iron.

The cells are then rinsed with PBS 1× in an amount of 100 μL per well, and then incubated for 30 min at 37° C. in a humid atmosphere with 5% CO₂ and with 100 μL of PBS 1×.

These 1 h 30 minutes of incubation at 37° C. allow the intracellular esterases to deacetylate the H₂DCF probe, oxidizable by intracellular OASes into DCF; a fluorescent compound, the formation of which is proportional to the amount of intracellular OASes.

The fluorescence intensity is read with the cytofluorimeter at λ_{excitation}=485 nm and λₑₘᵢₛₛᵢₒₙ=530 nm. It reflects the amount of produced intracellular OASes.

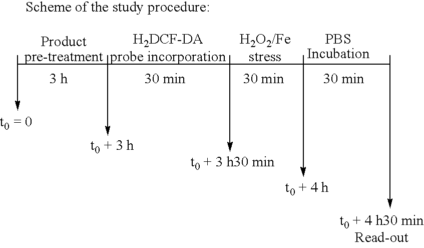

### Calculation of the % of Protection Against Intracellular Production of OASes

With the below ratio, it is possible to calculate for each concentration of tested product the % of protection against production of intracellular OASes (since the fluorescence intensity or FI expresses intracellular release of OASes).

$$\frac{[ FI Oxidant ( {H}_{2} {O}_{2} / Fe ) - FI ( Oxidant + Product ) ] \times 100}{FI Oxidant ( {H}_{2} {O}_{2} / Fe ) - FI Control}$$

The values indicated in Table 3 are percentages of inhibition of intracellular OAS production following an exogenous oxidative stress, relatively to <<control>> cells (100%) and to <<stimulated>> cells (0%).

The protective mean percentages, for the 13 tested molecules at 4 concentrations on the L929 line are presented in Table 3 below.

TABLE 3

| | | | | | | Doses | | | |
|---|---|---|---|---|---|---|---|---|---|
| | Compounds of formula (I) | | | | | 0.02 | 0.2 | 2 | 20 |
| No. | n | R | Rn | R1 | Salt | ng/mL | ng/mL | ng/mL | ng/mL |
| 1 | 2 | H | H | H | HCl | 12 | 13 | 5 | 1 |
| 2 | 2 | H | H | F | HCl | 14 | 19 | 14 | −13 |
| 3 | 2 | H | H | F | HCl | 15 | 11 | 5 | 1 |
| 4 | 3 | H | H | H | HCl | 13 | 12 | 8 | −14 |
| 5 | 3 | H | H | F | HCl | 18 | 23 | 26 | 23 |
| 6 | 3 | H | H | F | HCl | 31 | 30 | 21 | 26 |
| 7 | 4 | H | H | H | HCl | 31 | 33 | 26 | 32 |
| 8 | 5 | H | H | H | HCl | 22 | 25 | 20 | 19 |
| 9 | 5 | H | H | F | HCl | 18 | 20 | 23 | 18 |
| 10 | 10 | H | H | F | HCl | 20 | 25 | 18 | 15 |
| 11 | 10 | H | H | H | HCl | 20 | 19 | 22 | 19 |

| | |
|---|---|
| Reference anti-radical product | Dose = 400 μg/mL |
| Vitamin E | 31 |

### Conclusion:

In vitro, at a cellular scale, an exogenous stress by H₂O₂/Fe²⁺-Fe³⁺ is capable of inducing intracellular OAS production detected by the fluorescent probe.
Vitamin E (reference anti-radical molecule) has an average protection of 31% at 400 μg/mL.
The short chain (C₇-C₉) unsaturated fatty amino-acid derivatives according to the invention in particular the compounds Nos. 8, 7 and 6, have anti-radical properties (activity >25%).

5) Study of the Anti-Radical Effect. Analysis of Lipid Peroxidation

Moreover, during normal cellular metabolism, upon occasional exposure of the skin to stressing agents or during dermatological pathologies, reactive oxygenated species further called Free Oxygenated Radicals (FOR) are generated (Y. M. W. Janssen et al., 1993). These FORs described as very reactive metabolites, play an important role in a great number of processes such as inflammation, ageing and tumoral promotion.

FORs are considered as “second messengers” in cellular signaling of the oxidative stress and therefore as early mediators of inflammation (A. Van Der Vliet and A. Bast, 1992).

Their overproduction induces substantial damages within the cell. Certain cellular constituents are then major targets of such an oxidative stress: the lipid components of the plasma membrane (lipoperoxidation), proteins (denaturation and degradation) and the genetic material or DNA (mutations) may be altered. The cells are capable of limiting these oxidative damages by different anti-radical defense systems (enzymatic and non-enzymatic antioxidants) (B. P. Yu, 1994; H. Steiling et al, 1999).

However, under certain conditions, FORs are produced in such an amount that the cellular antioxidant activity is insufficient; these FORs then become factors inducing inflammatory pathologies and ageing of tissues (Y. Miyachi and al., 1986; M. Kress et al., 1995).

In order to enhance anti-radical activity of the different derivatives according to the invention, we have analyzed their protective power against alteration of cell membranes induced by an oxidative (chemical) stress as compared with a reference antioxidant, vitamin E.

The plasma membrane forms the main and primary target of the FORs and, being rich in lipids, it is the site of increased peroxidation (A. W. Girotti, 1985). The generated peroxides during this lipid oxidation are also very reactive and capable of degrading the protein and genomic material.

In order to evaluate membrane alteration, we have measured lipid peroxidation by an assay in vitro of the complexes between lipid oxidation products and thiobarbituric acid. These complexes are called TBARS (Thiobarbituric Acid Reactive Substances) and give the name to the test: Test of TBARS.

In order to mimic a chemical oxidative stress, we treated a fibroblast line, L929, with a complex composed of hydrogen peroxide (H₂O₂) and of iron (Fe²⁺/Fe³⁺) thereby regenerating Fenton's reaction, source of FORs and more particularly of hydroxyl radicals)(OH⁰) (D. A. Vessey et al., 1992):

H₂O₂+Fe²⁺->OH⁰+OH⁻+Fe³⁺

The products were evaluated on the murine fibroblast line L929. The cells were pretreated with the different concentrations of products for 16 hours and were then stimulated with the complex H₂O₂—Fe²⁺/Fe³⁺ (200 μM-1 mM) for 1 hour.

Mother solutions: 100 mg/mL, ethanol, 4° C.
Final solutions: 0.02 ng/mL.

Peroxidation of membrane lipids is analyzed by measuring the TBARS (Procedure Ref. PL No. 2, according to Morlière et al., 1991).

Principle of the Test:

In an acid medium, at 95° C., complexes noted as TBARS for Thio Barbituric Acid Reactive Substance are formed, between the lipid oxidation products (malonic dialdehyde or MDA) and thiobarbituric acid (TBA) which may be assayed by fluorescence relatively to a standard range with MDA. The dosage of the TBARS is then expressed in pmol/μg of proteins. The proteins and the TBARS are assayed in the intracellular medium.

Calculation of the Percentage of Protection of Cell Membranes:

From the calculation of the TBARS in pmol/μg of proteins, we calculated the protective efficiency of different products against oxidation of membrane lipids.

$$\frac{\% of protection \text{:} \begin{matrix}[ TBARS Control ] - \\ [ TBARS ( + products ) ]\end{matrix}}{TBARS control} \times 100$$

Seven independent experiments were conducted. During these experiments, various compounds were evaluated (with the test, it is not possible to evaluate more than 10 molecules at the same time). The compounds which were evaluated several times were selected according to the results obtained in the other test also measuring antiradical activity (test for assaying oxygen active species, OASes).

The model used in this experiment (Fenton reaction) induces significant lipid peroxidation in L929 fibroblasts. This massive discharge of OH⁻ hydroxyl radicals therefore generates an oxidative stress at cell level and notably at membrane level. However, in this type of oxidative reaction, the products from lipid peroxidation are internalized in the cells and the TBARS are then assayed in the intracellular medium.

Vitamin E at 400 μg/mL, reduces the lipid peroxidation induced by the complex H₂O₂—Fe²/Fe³, and very effectively protects cell membranes.

In the following Table 4, the results of 7 experiments are shown.

TABLE 4

| | | | | | | % of protection of | |
|---|---|---|---|---|---|---|---|
| | | | | | | membrane lipids | |
| Compounds according to | | | | | | Dose = 0.02 ng/mL | |
| the invention | | | | | | Number of | |
| No. | n | R | Rₙ | R₁ | Salt | experiments | Average |
| 1 | 2 | H | H | H | HCl | 3 | 13.41 |
| 2 | 2 | H | H | F | HCl | 3 | 7.63 |
| 3 | 2 | H | H | F | HCl | 3 | −4.00 |
| 4 | 3 | H | H | H | HCl | 3 | 8.75 |
| 5 | 3 | H | H | F | HCl | 3 | 36.27 |
| 6 | 3 | H | H | F | HCl | 5 | 49.82 |
| 7 | 4 | H | H | H | HCl | 4 | 48.30 |
| 8 | 5 | H | H | H | HCl | 5 | 56.61 |
| 9 | 5 | H | H | F | HCl | 3 | 27.49 |
| 10 | 10 | H | H | F | HCl | 3 | 23.91 |
| 11 | 10 | H | H | H | HCl | 2 | −26.31 |

### Conclusion:

The in vitro model presented in this study reflects the consequences due to major oxidative stress on the main cell target which is the plasma membrane. Thus, the assay of the lipid peroxidation is a good marker of the oxidative stress and allows evaluation of the antioxidant action towards the hydroxyl radical, of active ingredients at the cell membrane.

Under our experimental conditions, we observe that compounds Nos. 8, 9, 10, 7, 5 and 6 have significant antiradical activity and very effectively protect the membranes of the cells.

Compounds Nos. 8, 7 and 6 have the most significant antiradical activity and protection of the membranes.

### 6) EFFECT OF THE UNSATURATED FATTY AMINO-ACID DERIVATIVES ACCORDING TO THE INVENTION ON THE SYNTHESIS OF MELANIN IN VITRO

Melonocytes are cells with a star aspect, present in minority in the basal layer of the epidermis. Their main function is to provide melanogenesis, a process by which melanin is synthesized in specialized organelles, the melanosomes, and then transported and distributed to the neighboring keratinocytes via their dendritic extensions. This contact with keratinocytes allows skin pigmentation, a mechanism for protecting the epidermis against the mutagenic effects of ultraviolet rays. Each melanocyte is connected with about 36 keratinocytes, thereby forming an epidermal melanization unit.

Melanogenesis consists in a series of enzymatic and spontaneous reactions, the precursor of which is tyrosine. Three main enzymes are involved in this process: tyrosinase, and tyrosinase-related protein 1 and 2 (TRP 1 and 2) (Jimbow et al., 2000). Tyrosinase catalyzes the transformation of tyrosine into dopaquinone. From there, two synthesis routes are then possible: eumelanogenesis and pheomelanogenesis. Conversion of dopaquinone into eumelanin is accomplished by a series of successive oxidation reactions involving TRP-1 and TRP-2. Eumelanin corresponds to the black brown pigment with low sulfur content and ensures photoprotective power. In pheomelanogenesis, high sulfur content molecules are incorporated to dopaquinone in order to provide pheomelanin of yellow-orange color, present in the skins of redheads.

The physiological stimulus of the synthesis of melanin is the sun, which causes an increase in the number of melanocytes, neosynthesis of melanin, and morphological modifications of the melanocytes, associating an increase in their dendricity with an increase of the transfer of melanosomes to keratinocytes. At a molecular level, exposure to the sun stimulates synthesis and secretion of alpha-melanocyte stimulating hormone. α-MSH increases the intramelanocytary AMPc concentration, activating the transcription factor, Mitf, which in turn stimulates transcriptional activity of the genes coding for tyrosinase, TRP-1 and TRP-2.

Certain exogenous molecules are also known for negatively regulating melanogenesis. Hydroquinone inhibits the synthesis of melanin by appearing as a substrate of tyrosinase in order to divert its activity (Curto et al., 1999). Vitamin C inhibits tyrosinase but also behaves as a powerful reducing agent by preventing coloration of melanin by oxidation.

6.1) The modulating effect of unsaturated fatty amino-acid derivatives on melanogenesis was analyzed. For this, a measurement of the synthesis of melanin by a colorimetric assay is carried out on a cell line of murine melanomas: the B16-F10 line.

The effect of the products is investigated in a basal situation and after stimulating melanogenesis by a MSH for determining the depigmenting power.

Assay of Melanin:

The level of extracellular and intracellular melanin is then measured by spectrophotometry at a wave length of 405 nm according to the procedure (Meun, Y. J. et al.). The amount of pigment is determined by a standard range of melanin and by analysis on the Microwin software package (Berthold Biotechnologies). An assay of total proteins is conducted for samples of intracellular melanin by the BCA-Copper method at 540 nm. The standard range is produced with a standard protein, BSA (Bovine Albumin Serum).

### 6.2) Results

In a basal situation, alpha MSH at 1 μM increases melanin production by more than 100% as compared with control cells.

This increase in melanin is countered by depigmenting agents, such as hydroquinone at 1 μg/mL, or vitamin C at 40 μg/mL which inhibit melanogenesis by about 60%.

De-Pigmenting Molecule:
At 30 μg/mL, the molecule No. 11 weakly inhibits the amount of intra- and extra-cellular melanin, by about 45% and by 30%, respectively. However, molecule No. 11 remains without any effect on the synthesis of melanin at 10 μg/mL.

Pigmenting Molecules:
At 3, 10 and 30 μg/mL, molecule No. 6 increases the amount of intracellular melanin by about 100%. Molecule No. 5 also seems to have a pigmenting effect, but it is weaker than that of molecule No. 6.

### 7) STUDY OF THE ANTI-COLLAGENASE ACTIVITY

In order to measure anti-collagenase activity, reference may notably be made to French patent application No. 2 829 491, published on March 14^{th} 2003.

### 7.1. On Frozen Human Skin Cuts

### 7.1.1. Procedure

This study is conducted on different solutions at 1% and 2% concentration of active ingredients as compared with the excipient alone, the buffer controls and collagenase. The active ingredients used are DHA, hydroxyl-10-decene-2(trans)oic acid 2-dimethylamino-ethyl ester (ML40) and hydroxyl-10-decene-2(trans)oic acid glycerol ester (GM).

Frozen 5 μm cuts, from mammary plasty of a women of 54 years of age, are placed on histological slides (4 cuts per slide). Each solution is tested on a slide.

The cuts are covered with the solution to be tested and incubated for 2 hours at 37° C. in a humid chamber. The solutions are removed by recurrent rinses and the cuts are colored with picrosirius. A microscopic examination is carried out.

### 7.1.2. Results

The products of the present invention have an activity which is greater than the activity of DHA known previously.

### 7.2. On Human Skin Explants Maintained Alive

### 7.2.1. Procedure

The study is conducted on a 5% GM product as compared with the excipient (hydrocerin), a positive control and a control in the presence of collagenase at 100 U/mL. Hydrocerin is used as an excipient for preparing the product to be applied.

This study was conducted twice. In the first study, it was noticed that the action of collagenase at day+2 remains very limited and not significant. In the second study, the application time is extended and the taking of explants is carried out on day+2 and on day+4.

### 7.2.1.1. Preparation of the Explants

Human skin explants prepared and distributed in 16 batches each with 3 explants are maintained alive according to Table 5:

TABLE 5

| | Day + 2 | Day + 4 |
|---|---|---|
| Control | 3 explants | 3 explants |
| Excipient | 3 explants | 3 explants |
| Product & 5% GM | 3 explants | 3 explants |
| Positive test | 3 explants | 3 explants |
| Control + collagenase | 3 explants | 3 explants |
| Excipient + collagenase | 3 explants | 3 explants |
| Product 2 5% GM + collagenase | 3 explants | 3 explants |
| Positive test + collagenase | 3 explants | 3 explants |

7.2.1.2. Application of the Product at 5% of GM and of Products from the Present Invention

The product is applied on day0 and on day+2 in an amount of 20 mg per explant and collagenase is incorporated into the culture media of the last 24 batches.

### 7.2.1.3. Histology

Three explants of each batch are taken at day+2 and day+5 day+4 and fixed with ordinary Bouin liquid and treated in histology. The histological study comprises:
impregnation in paraffin
cuts, staining with red Sirius F3B
colorimetric measurement of collagen by image analysis
report with photographs.

### 7.2.2. Results

Samples carried out on day2 do not show any significant activity of collagenase regardless of the examined batch. For this reason, survival, contact and application are extended up to day+4. The action of collagenase is noted in 2 ways: intensity of the coloration of the collagen network and thickness of the dermal structure. With this study, a correlation is performed between the penetration of the active ingredient and its inhibitory activity towards collagenase. The obtained results are the following:
for controls without collagenase, the dermis has a normal structure with regular collagen bundles in all the compartments,
for controls with collagenase, the collagen bundles are very strongly degraded and the thickness of the dermis is reduced by half,
for explants with excipient and collagenase, the collagen bundles are strongly degraded, the alteration being less than that observed on controls with collagenase, and the thickness of the dermis is reduced by almost half.

By comparison with the GM product, the products from the invention show a superior and more rapid anti-collagenase activity than the esters of DHA (GM).

### BIBLIOGRAPHY

Janssen Y, M, W, et al. (1993) Lab, Invest, 69:261-74
Van Der Vliet A, and Bast A, (1992) Chem, Biol, Interactions 85: 95-116
Yu B, P, (1994) Physiol, Rev, 74: 139-62
Steiling H, et al. (1999) Exp. Cell, Res, 247:484-94
Miyachi Y, et al. (1986) J, Clin, Lab, Immunol, 19: 11-4
Kress M, et al. (1995) Pain 62: 87-94
Girotti A, W, (1985) J, Free Radic, Biol, Med, 1: 87-95
Vessey D, A et al. (1992) J, Invest, Dermatol, 99: 859-63
Morlière P, et al, (1991) Biochim, Biophys, Acta, 1084:261-268
Emonet E et al. (1997) J. Photochem. Photobiol 40: 84-90
Meun, Y. J. et al. (2004) Biol. Pharm. Bull. June, 27 (no. 6): 806-809
Curto EV., Kwong C., Hermersdörfer H., Glatt H., Santis C., Virador V., Hearing VJ., Dooley TP. Inhibitors of mammalian melanocyte tyrosinase: in vitro comparisons of alkyl esters of gentisic acid with other putative inhibitors (1999) Biochemical Pharmacology 57: 663-672
Jimbow K., Hua C., Gomez P., Hirosaki K., Shinoda K., Salopek T., Matsusaka H., Jin H., Yamashita T. 1ntracellular vesicular trafficking of tyrosinase gene family protein in eu- and pheomelanosome biogenesis (2000) Pigment Cell Res. 13: 110-117

1. An unsaturated fatty amino-acid derivative, as drugs, fitting the general formula (I): 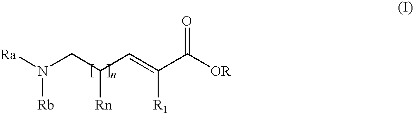
as well as their pharmaceutically acceptable acid addition salts, wherein:
Rn represent independently of each other a hydrogen atom or a linear or branched alkyl group comprising 1-6 carbon atoms, and being optionally substituted with one or more halogen atoms;
R₁ represents a hydrogen, fluorine, chlorine or bromine atom, or else a —CF₃ or —CHF₂ group or else a linear or branched alkynyl, alkenyl or alkyl group comprising 1-6 carbon atoms, and being optionally substituted with one halogen atom;
R represents a hydrogen atom or a group R′ representing a linear or branched alkyl group comprising 1-6 carbon atoms, or a cycloalkyl group comprising 3-6 carbon atoms, and being optionally substituted with one halogen atom;
Ra and Rb represent independently of each other a hydrogen atom or a linear or branched alkyl or acyl group comprising 1-6 carbon atoms, Ra and Rb may form together a hydrocarbon cycle including 4-6 carbon atoms; and
n is an integer comprised between 2 and 14.

2. The unsaturated fatty amino-acid derivatives of general formula (I) according to claim 1, wherein Ra and Rb represent a hydrogen atom.

3. The unsaturated fatty amino-acid derivative of general formula (I) according to claim 1, wherein R represents a hydrogen atom.

4. The unsaturated fatty amino-acid derivative of general formula (I) according to claim 1, wherein Rn represents a hydrogen atom.

5. The unsaturated fatty amino-acid derivative of general formula (I) according to claim 2, wherein R₁ represents a hydrogen or fluorine atom.

6. The unsaturated fatty amino-acid derivative of general formula (I) according to claim 2, wherein n is comprised between 2 and 10.

7. The unsaturated fatty amino-acid derivative of general formula (I) according to claim 2, wherein n is comprised between 3 and 5.

8. The unsaturated fatty amino-acid derivative of general formula (I) according to claim 2, wherein n is comprised between 9 and 14.

9. The unsaturated fatty amino-acid derivative of general formula (I) according to claim 2, wherein n is equal to 3.

10. The unsaturated fatty amino-acid derivative of general formula (I) according to claim 1, wherein they are in the form of Z or E isomers, or of a mixture of Z isomers and of E isomers, in any amounts.

11. The unsaturated fatty amino-acid derivative of general formula (I) according to claim 1, wherein they are in the form of pharmaceutically acceptable acid salts formed from hydrochloric acid, hydrobromic acid, sulfuric acid, nitric acid, phosphoric acid, acetic acid, benzene-sulfonic acid, benzoic acid, camphor-sulfonic acid, citric acid, ethane-sulfonic acid, fumaric acid, glucoheptonic acid, gluconic acid, glutamic acid, glycolic acid, hydroxynaphthoic acid, 2-hydroxyethane sulfonic acid, lactic acid, maleic acid, malic acid, mandelic acid, methane-sulfonic acid, muconic acid, 2-naphthalene-sulfonic acid, propionic acid, salicylic acid, succinic acid, dibenzoyl-L-tartaric acid, tartaric acid, p-toluene-sulfonic acid, trimethylacetic acid or trifluoroacetic acid.

12. The unsaturated fatty amino-acid derivative of general formula (I) according to claim 1, wherein they are selected from:
(E)-6-amino-hex-2-enoic acid hydrochloride,
(E)-7-amino-hept-2-enoic acid hydrochloride,
(E)-8-amino-oct-2-enoic acid hydrochloride,
(E)-9-amino-non-2-enoic acid hydrochloride,
(E)-10-amino-dec-2-enoic acid hydrochloride,
(E)-14-amino-tetradec-2-enoic acid hydrochloride,
6-amino-2-fluoro-hex-2-enoic acid hydrochloride as a mixture of isomers Z and E,
(Z)-6-amino-2-fluoro-hex-2-enoic acid hydrochloride,
(E)-6-amino-2-fluoro-hex-2-enoic acid hydrochloride,
7-amino-2-fluoro-hept-2-enoic acid hydrochloride as a mixture of isomers Z and E,
(Z)-7-amino-2-fluoro-hept-2-enoic acid hydrochloride,
9-amino-2-fluoro-non-2-enoic acid hydrochloride,
10-amino-2-fluoro-dec-2-enoic acid hydrochloride and
14-amino-2-fluoro-tetradec-2-enoic acid hydrochloride.

13. An unsaturated fatty amino-acid derivative, as novel chemical compounds, fitting general formula (I) as defined according to claim 1, the following compounds being excluded:
6-amino-hex-2-enoic acid, its hydrochloride and its trifluoroacetate, 8-amino-oct-2-enoic acid trifluoro-acetate, 8-(dimethylamino)-oct-2-enoic acid, 12-(dimethyl-amino)-dodec-2-enoic acid, ethyl 6-(isopropylamino)-2-methyl-hex-2-enoate, ethyl 6-(tert-butylamino)-2-methyl-hex-2-enoate, methyl 6-amino-2-methyl-hex-2-enoate, methyl 7-amino-hept-2-enoate, methyl 7-amino-2-methyl-hept-2-enoate and methyl 7-amino-4-methyl-hept-2-enoate.

14. A dermatological compositions comprising as an active ingredient, at least one unsaturated fatty amino-acid derivative according to claim 1, in association with a dermatologically acceptable excipient.

15. A method for treating skin ageing and/or intended for treating keratinisation and pigmentation disorders, and/or intended to improve healing comprising the application of a dermatocosmetological composition comprising an unsaturated fatty amino-acid derivative according to claim 1 to a person in need thereof.

16. The method according to claim 15, wherein said method is intended for treating psoriasis, prurit, and/or atopic dermitis.

17. The method according to claim 15, wherein said method is intended for repigmenting hair or skin, notably white age spots.

18. The method according to claim 15, wherein said method is intended for causing lightening of the skin or for treating brown age spots.

19. A method for preparing an unsaturated fatty amino acid derivative of the following general formula (I): 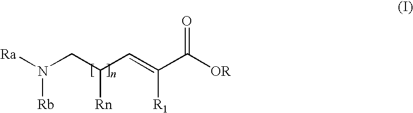
wherein:
Rn represent independently of each other a hydrogen atom or a linear or branched alkyl group comprising 1-6 carbon atoms, and being optionally substituted with one or more halogen atoms;
R₁ represents a hydrogen, fluorine, chlorine or bromine atom, or else a —CF₃ or —CHF₂ group or else a linear or branched alkynyl, alkenyl or alkyl group, comprising 1-10 carbon atoms, and being optionally substituted with a halogen atom;
R represents a hydrogen atom or a group R′ representing a linear or branched alkyl group comprising 1-6 carbon atoms, or a cycloalkyl group comprising 1-6 carbon atoms, and being optionally substituted with a halogen atom;
Ra and Rb represent independently of each other a hydrogen atom or a linear or branched alkyl or acyl group comprising 1-6 carbon atoms, Ra and Rb may form together a hydrocarbon cycle including 4-6 carbon atoms; and
n is an integer comprised between 2 and 14;
said preparation method comprising:
the application of the Wittig-Horner reaction by reacting a phosphonate of the following general formula (II): 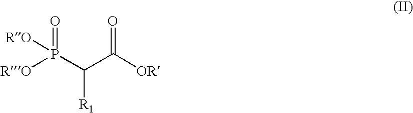
wherein:
R₁ is as defined above,
R′ represents a linear or branched alkyl group, comprising 1-6 carbon atoms, and
R″ and R″′ represent independently of each other, a linear or branched alkyl group, comprising 1-6 carbon atoms, said groups R″ and R″′ may form an hydrocarbon cycle comprising 2-4 carbon atoms,
or, when R₁═H, the application of the Doebner-Knoevenagel reaction by reacting a malonic acid derivative of formula R′—OOC—CH₂—COOR′, R′ being as defined above,
on a compound of the following formula (III): 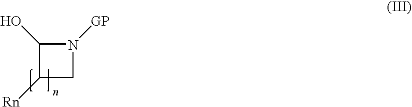
wherein:
n is as defined above, and
GP represents a protective group,
in order to obtain a compound of the following formula (IV): 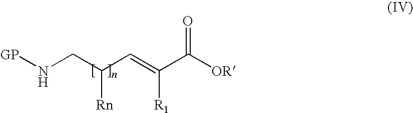
for which GP, R′, R₁, Rn and n are as defined above,
an optional saponification reaction of the compound of the aforementioned general formula (IV), in order to obtain a compound of the following general formula (V): 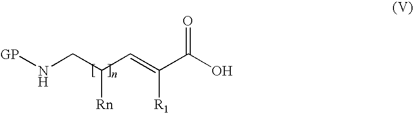
for which GP, R₁, Rn and n are as defined above,
a reaction for deprotecting the nitrogen of the compound of the aforementioned formula (IV) or (V), in order to obtain a compound of formula (I) wherein Ra and Rb represent a hydrogen,
and an optional N-alkylation or N-acylation reaction of the preceding compound of formula (I) wherein Ra and Rb represent a hydrogen, in order to obtain a compound of formula (I) wherein at least one of the Ra and Rb groups does not represent a hydrogen.

20. The method according to claim 19, wherein the compound of general formula (III): 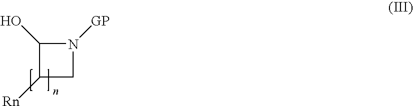
is synthesized from a compound of general formula (VI) 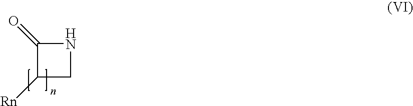
wherein n and Rn are as defined in claim 19
according to the following steps:
protection of the nitrogen of the amide function of the compound of formula (VI) with a GP group in order to obtain a compound of the following general formula (VII): 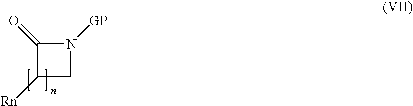
wherein n, Rn and GP are as defined in claim 19,
and then reduction of the carbonyl function of the compound of formula (VII) as defined earlier.

21. The method according to claim 19, wherein Ra and Rb represent a hydrogen.

22. The method according to claim 19, wherein R represents a hydrogen.

23. The method according to claim 19, wherein Rn represent a hydrogen.

24. The method according to claim 19, wherein R₁ represents a hydrogen or a fluorine.

25. As synthesis intermediates, a compound of general formula (VIII): 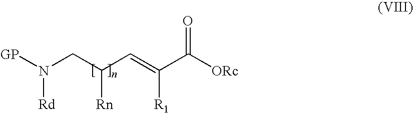
for which:
Rc represents a hydrogen or a linear or branched alkyl group comprising 1-6 carbon atoms,
R₁ represents a hydrogen, fluorine, chlorine or bromine atom, or else a —CF₃ or —CHF₂ group or else a linear or branched alkynyl, alkenyl or alkyl group comprising 1-6 carbon atoms, and being optionally substituted with one or more halogen atoms;
Rn represent independently of each other a hydrogen atom or a linear or branched alkyl group comprising 1-6 carbon atoms, and being optionally substituted with one or more halogen atoms;
n is an integer comprised between 2 and 14.
GP represents a protective group,
Rd represents a hydrogen or a protective group GP′;
the following compounds being excluded: tert-butyl(E)-6-(N,N-di-tert-butoxy-carbonylamino)-hex-2-enoate, ethyl(E)-6-(N-tert-butoxy-carbonylamino)-hex-2-enoate, methyl (E)-7-(N-tert-butoxy-carbonylamino)-hept-2-enoate, and (E)-7-(N-tert-butoxy-carbonylamino)-hept-2-enoic acid.

26. The compound according to claim 25, wherein Rn represent a hydrogen.

27. The compound according to claim 26, wherein R₁ represent a hydrogen or a fluorine.

28. The method according to claim 19, wherein the protective group GP forms with the adjacent nitrogen atom a group of the carbamate type.

29. The method according to claim 28, wherein the protective group GP forms with the adjacent nitrogen atom a tert-butyl or benzyl carbamate.

30. The method according to claim 20, wherein the reduction of the carbonyl function of the compound of formula (VII) is carried out with aluminium hydrides such as diisobutyl aluminium hydride at low temperature.

31. The compound according to claim 25, wherein the protective group GP forms with the adjacent nitrogen atom a group of the carbamate type.

32. The compound according to claim 32, wherein the protective group GP forms with the adjacent nitrogen atom a tert-butyl or benzyl carbamate.

33. The compound according to claim 25, wherein N-GP′ represents a tert-butyl or benzyl carbamate.
